(19) United States
(12) Patent Application Publication
(10) Pub. No.: US 2003/0158118 A1
(43) Pub. Date: Aug. 21, 2003

(54) Combination of cimetidine and cysteine derivatives for treating cancer

(76) Inventor: Morten Sloth Weidner, Virum (DK)

Correspondence Address: BIRCH STEWART KOLASCH & BIRCH, PO BOX 747, FALLS CHURCH, VA, 22040-0747, US

(21) Appl. No.: 10/303,867

(22) Filed: Nov. 26, 2002

Related U.S. Application Data
(60) Provisional application No. 60/395,344, filed on Jul. 12, 2002.

(30) Foreign Application Priority Data
Nov. 26, 2001 (DK) PA 2001 01761
Jul. 10, 2002 (DK) PA 2002 01086

(51) Int. Cl. A61K 38/06; A61K 38/04; A61K 31/4172; A61K 31/198

(52) U.S. Cl. 514/17; 514/18; 514/400; 514/562

(57) ABSTRACT

The present invention relates to new substances in the form of chemical complexes comprising cimetidine or a derivative thereof and a cysteine derivative and to compositions comprising said complexes or combination. The invention further relates to the therapeutic effect of such combinations in relation to treating cancer, cancer chemoprevention or the suppression of hypersensitivity and/or inflammatory reactions of a mammal.

### FIELD OF THE INVENTION

The present invention relates to a chemical complex comprising cimetidine or a derivative thereof and a cysteine derivative. The combination of cimetidine or a derivative thereof and a cysteine derivative in the preparation of a pharmaceutical product for the treatment of cancer, chemoprevention or the suppression of hypersensitivity and inflammatory reactions of a mammal is disclosed herein.

### BACKGROUND OF THE INVENTION

A number of drug classes are available for the treatment of cancer. Unfortunately, such drugs are associated with a number of serious side effects, e.g. immunosuppression.

Cancer is caused by an uncontrolled proliferation of cells that express varying degrees of fidelity to their precursors. These cancer cells form a malignant tumour that enlarges and may spread to adjacent tissues or through blood and lymph systems to other parts of the body. There are numerous forms of cancer of varying severity. For most types of cancer there is no effective treatment today. Some pharmaceuticals and dietaries have been mentioned as relevant in the treatment of cancer including anti-inflammatory agents; vitamin A, C, D, E; β-carotenes; folic acid; N-acetylcysteine; and H2-antagonists (Langham and Boyle, Chemoprevention of colorectal cancer, Gut 1998, 43: 578-585).

N-acetylcysteine is a drug substance, which has been widely used as a mucolytic and as an antidote to acetaminophen poisoning.

Cimetidine is one of the most widely used drugs in the world. The presently primary pharmacological action is mediated through antagonism of histamine H2 receptors for which reason cimetidine is widely used for treating gastritis. However, other pharmacological actions of cimetidine are known. For example it was found that cimetidine enhances the hepatoprotective action of N-acetylcysteine in mice treated with toxic doses of paracetamol (Zaki H. Al-Mustafa, et al: Cimetidine enhances the hepatoprotective action of N-acetylcysteine in mice treated with toxic doses of paracetamol, Toxicology 121 (1997) p 223-228). In this study, cimetidine in the form of Tagemet® and N-acetylcysteine in the form of Mucomyst© were administered intraperitoneally to the mice, Furthermore, cimetidine has been used in a diagnostic method for selecting cancer patients for treatment with cimetidine (U.S. Pat. No. 6,268,156).

Cimetidine and cysteine derivatives have been used for many purposes. For example, cimetidine and N-acetyl-cysteine are mentioned as co-agents in the treatment of neurological diseases with 4-amino-benzoic acid and derivatives thereof (WO 95/01096). Furthermore, cimetidine and cystine have been used in combination with leukotriene antagonists, in particular montelukast, for combating inflammatory diseases (U.S. Ser. No. 2002/0137785).

Cimetidine in combination with pyridine carboxy derivatives (e.g. niacinamide) have been reported for having immunomodulating properties (WO 01/74780).

### SUMMARY OF THE INVENTION

The present inventor has found that a combination of cysteine or cysteine derivatives and cimetidine or a derivative thereof has tumor-suppressing activities in mammals, such as in humans.

Such a combination is advantageously provided in the form of a chemical complex comprising one or more of such cysteine derivative(s) and cimetidine and/or one or more cimetidine derivatives. Obviously, the combination may also be provided in the form of a pharmaceutical composition comprising such a combination, optionally in the form of a complex, and one or more acceptable excipients and carriers.

Accordingly, the present inventor has recognised the therapeutic activity of a combination of cysteine derivatives and cimetidine or a derivative thereof, for which reason the said combination, in particular in the form of a chemical complex, may be regarded as an active therapeutic agent.

Contrarily to existing therapeutic agents used in the treatment of cancer, the chemical complexes and compositions according to the present invention have the advantage of not being likely to be associated with any serious side effects, as all of their components are known to living organisms and are acknowledged as non-toxic and well-tolerated by the organism. The present inventor puts forward the hypothesis that the very beneficial therapeutic index exhibited by the complex and compositions of the invention is superior to the use of the individual constituents of the complex, and this may be due to synergistic effects and/or lower toxic load.

Accordingly, the present invention provides a chemical complex and a pharmaceutical composition comprising:

i) cimetidine or a derivative thereof according to Formula II and/or salts thereof as defined herein; and

ii) a cysteine derivative of Formula I, stereoisomers thereof and/or salts thereof as defined herein;

said composition further comprises one or more acceptable excipients or carrier(s).

An important aspect of the invention relates to the use of a combination of cimetidine or a derivative thereof of Formula II as defined herein and a cysteine derivative of formula I as defined herein for the preparation of a medicament for the treatment of cancer, chemoprevention and/or immunomodulation of a mammal, such as a human, as well as to a method for the treatment of cancer, chemoprevention and/or immunomodulation in a mammal, such as a human, comprising administration to said mammal of an effective amount of a combination of cimetidine or a derivative thereof and a cysteine derivative or pharmaceutically acceptable salts thereof, or administration of a chemical complex comprising said combination or said salts to said mammal.

The comprexes and compositions of the invention may have particular relevance for the treatment of cancers of the gastrointestinal system, e.g. colon cancer, rectal cancer, colorectal cancer, pancreatic cancer, stomach (gastric) cancer, oesophageal cancer, liver cancer or bladder cancer. Furthermore, the complexes and compositions of the invention may have a therapeutic potential in metastatic as well as invasive cancers, e.g. breast cancer, cancer of the male and female genital tract, cancer of the thymus, lung, stomach, small intestine, prostate, adrenal gland, pancreas, colon, lymphoid tissue, liver, brain, salivary gland, spleen and skin.

### DETAILED DESCRIPTION OF THE INVENTION

The present inventor provides data herein indicating that a combination, a chemical complex, of cimetidine and N-acetyl-cysteine significantly reduces the tumor growth of colorectal cancer cells. The results were shown in a widely acknowledged test model involving tumor progression in SCID mice xenografted with SW620 colorectal cancer cells and in BALB/c mice grafted with syngenic CT26 colorectal cancer cells. It was surprisingly found that the mean tumor size in the SCID mice xenografted with SW620 colorectal cancer cells was inhibited by 89% and 73% following 19 days and 22 days of treatment respectively. Furthermore, tumor growth was inhibited by about 77% in the in BALB/c mice grafted with syngenic CT26 colorectal cancer cells following 33 days of treatment with a complex consisting of cimetidine and N-acetyl-cysteine (see examples 3 and 4). The overall effect of the said combination yielded a 185% higher inhibition of tumor size than the sum of the inhibition of the components administered individually. That is to say that the combination resulted in a synergistic effect of about 3 times in relation to that of the individual compounds.

Thus, the present inventor has provided evidence that the combination of a cysteine derivative of Formula I and cimetidine or a derivative thereof of Formula II reduces the growth of colon cancer cells, even in a synergistic manner. It is further contemplated that complexes and compositions of the invention can reduces growth of cancer cells of various cellular origins, e.g. carcinomas that are cancers of epithelial origin and sarcomas that are cancers of mesenchymal origin.

The complexes and compositions of the invention have particular relevance for the treatment of cancers of the gastrointestinal system, e.g. colon cancer, rectal cancer, colorectal cancer, pancreatic cancer, stomach (gastric) cancer, oesophageal cancer, liver cancer or bladder cancer.

Furthermore, the complexes and compositions of the invention have a therapeutic potential in metatstatic as well as invasive cancers, e.g. breast cancer, cancer of the male and female genital tract, cancer of the thymus, lung, stomach, small intestine, prostate, adrenal gland, pancreas, colon, lymphoid tissue, liver, brain, salivary gland, spleen and skin. Non-limiting examples of cancers are described infra.

According to the invention, the combination of a cysteine derivative of Formula I and cimetidine or a derivative thereof of Formula II may be provided in the form of a chemical complex, in the form of a composition comprising said complex and optionally one or more acceptable excipient(s) or carrier(s), or in the form of a pharmaceutical composition comprising said combination. Moreover, the cysteine derivative of Formula I and cimetidine or a derivative thereof of Formula II may each be provided in separate compositions such as in the form of separate dosage units. It is further anticipated that the complexes and compositions of the invention may comprise mixtures of cysteine and/or cysteine derivatives, mixtures of cimetidine and/or derivatives thereof.

Without being limited to a particular theory, advantageously, said combination is provided in the form of a chemical complex for purposes of achieving a homogeneous mixture of the two agents, which may positively affect the resulting therapeutic effect.

The present inventor proposes the hypothesis that the very advantageous therapeutic index of the combination of cimetidine or a derivative thereof of Formula II and a cysteine derivative of Formula I in comparison to the individual therapeutic effect is due to the synergistic effects between the components of the composition. Therefore, lower doses of one or both types of agents may be needed for providing the therapeutic effect, resulting in a lower toxic load on the body in comparison to the individual compound, while still achieving a surprisingly good therapeutic effect.

The invention is based, at least in part, on the synergistic activity of a cysteine derivative with cimetidine or a derivative thereof in comparison to either component. As stated, the cimetidine is classified as a histamine H2 receptor antagonist, which may be an important property in relation to the anticancer effect of cimetidine. Therefore, the surprising synergism of a cysteine derivative and/or cimetidine or a,derivative thereof allows for the combining of any derivative of cimetidine, which exhibits histamine H2 receptor antagonism and/or anticancer properties with a cysteine derivative to achieve the desired effect.

Accordingly, the present invention provides in a first aspect a substance consisting of a chemical complex and in a second aspect a pharmaceutical composition, said chemical complex and said composition comprising:

i) cimetidine or a derivative thereof of Formula II and/or salts thereof, as defined herein; and

ii) a cysteine derivative of Formula I, stereoisomers thereof and/or salts thereof, as defined herein,

wherein said composition further comprises one or more acceptable excipients.

As used herein, the phrase a substance consisting of a chemical complex is intended to mean a chemical entity consisting of the said combination of cimetidine or derivatives thereof and a cysteine derivative, optionally the chemical complex may further comprise pharmaceutically acceptable excipients, solvent residues and/or one or more therapeutically active agent(s).

Derivatives of Cimetidine

As used herein the term “cimetidine or a derivative thereof” is denoted to include cimetidine, salts of cimetidine, pro-drugs, and metabolites of cimetidine as well as derivatives of cimetidine, which may be in the form of salts and/or stereoisomers. Furthermore, it should be understood that the invention comprehends the different derivatives of cimetidine, salts of cimetidine, salts of cimetidine derivatives and cimetidine in isolation from each other, as well as mixtures of cimetidine, salts and/or derivatives of cimetidine.

A derivative of cimetidine is defined according to formula II:

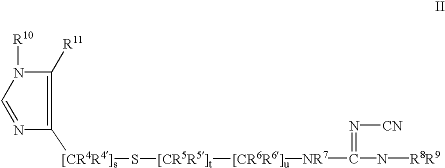

wherein s is a whole number from 1-3, t is a whole number from 0-2 and u is a whole number from 1-2;

R⁴, R^{4′}, R⁵, R^{5′}, R⁶, R^{6′} are each a monoradical independently selected from the group consisting of hydrogen, halogen, hydroxyl, C₁-C₆-alkylene, C₁-C₈-acylene and arylene;

R⁷, R⁸, R⁹, R¹⁰ are each a monoradical independently selected from the group consisting of hydrogen, nitro, C₁₋₆-alkylene, C₂-C₆-alkenyl, C₁₋₈-acylene and arylene; and

R¹¹ is a monoradical independently selected from the group consisting of hydrogen, halogen, hydroxy, C₁-C₆-alkylene, C₁-C₈-acylene and arylene.

The term “C₁-C₆ alkylene” is intended to mean a linear or branched saturated hydrocarbon chain wherein the longest chain has from one to six carbon atoms, such as methyl, ethyl, n-propyl, n-butyl, pentyl, or hexyl. A branched hydrocarbon chain is intended to mean a C₁₋₆-alkyl substituted at any carbon with a hydrocarbon chain such as isopropyl, isobutyl, sec-butyl, tert-butyl, isopentyl or neopentyl. The “C₁-C₆ alkylene” may optionally be substituted with an amino, a cyano, a hydroxy, a halogen, a nitro or a thiol group.

Likewise, the term “C₂-C₆-alkenylene” is intended to mean a linear or branched unsaturated hydrocarbon chain with one or more double bonding(s) wherein the longest chain has from two to six carbon atoms. The “C₂-C₆ alkenylene” may optionally be substituted with an amino, a cyano, a hydroxy, a halogen, a nitro or a thiol group.

The term “C₁-C₈-acylene” characterises an alkylene that is linear or branched, saturated or unsaturated, preferably monounsaturated, or an arylene, wherein said alkylene and arylene has a —C═O group. Non-limiting examples are formyl, acetyl and benzoyl. The “C₁-C₈-acylene” may optionally be substituted with a substituent selected from the group consisting of an amino, a cyano, a hydroxy, a halogen, a nitro, sulphono, sulphanyl and/or a thiol group.

The term “arylene” characterises a phenyl ring optionally substituted 1-3 times with C₁-C₃-alkylene, amino, carboxyl, cyano, hydroxy, halogen, nitro, thiol group, wherein the C₁-C₃-alkylene is optionally substituted with amino, a cyano, a hydroxy, a halogen, a nitro or a thiol group. Preferably, the arylene is monosubstituted or substituted twice.

The term “halogen” includes fluorine, chlorine, bromine and iodine.

In some embodiments, at least one of R⁴ or R^{4′} is hydrogen. In some embodiments, at least one of R⁵ or R^{5′} is hydrogen and in some other of similar embodiments, at least one of R⁶ or R^{6′} is hydrogen. Preferably, at least one of R⁴ or R^{4′}, at least one of R⁵ or R^{5′} and at least one of R⁶ or R^{6′} is hydrogen.

In further embodiments thereof or alternative embodiments, R⁷ is preferably hydrogen or C₁-C₄-alkylene. In still further embodiments thereof or other embodiments R⁸ is preferably hydrogen or C₁-C₄-alkylene. In further embodiments thereof or other embodiments, s, t, and u is 1. In some further embodiments as well as alternative embodiments R⁴, R^{4′}, R⁵, R^{5′}, R⁶ and R^{6′} is independently selected from the group consisting of hydrogen, halogen and/or C₁-C₄-alkylene, preferably hydrogen. C₁-C₄-alkylene characterise a linear or branched saturated hydrocarbon chain wherein the longest chain has from one to four carbon atoms and wherein the C₁-C₄ alkylene” is optionally substituted with a substituent selected from the group consisting of an amino, a cyano, a hydroxy, a halogen, and/or nitro group.

Furthermore, in presently interesting embodiments, R¹¹ is a monoradical selected from the group consisting of hydrogen, halogen and/or C₁-C₆-alkylene.

In still interesting embodiments, R⁹ is a monoradical selected from the group consisting of hydrogen, nitro, C₁₋₆-alkylene, C₁₋₈-acylene and arylene. Still more interesting embodiments is wherein R¹⁰ is a monoradical selected from the group consisting of hydrogen, nitro C₁₋₆-alkylene, C₁₋₈-acylene and arylene.

Some derivatives may be prepared by replacement of one or more hydrogens bounded to an amino group by N-alkylation with alkyl halides to form tertiary alkyl amines or quaternary ammonium salts, N-acylation with acyl chlorides, esters or acids to yield amides, nitrosation of secondary alkylamines to yield N-nitrosoamines, addition of aldehydes and ketones to secondary alkylamines to yield an enamine. Thus, some embodiments relate to derivatives of cimetidine that is selected from the group consisting of amides, tertiary alkylamines or quaternary ammonium salts.

As used herein, the term “cimetidine or derivatives thereof” also encompasses oxidation of the thiol group so as to form a sulfoxide. Thus, in one embodiment, the derivative is a cimetidine sulfoxide.

The term “cimetidine or derivatives thereof” further encompasses various enantiomeric, diastereomeric and tautomeric forms in the event where such exist. It will be understood that the invention comprehends the different enantiomers, diastereomers and tautomers in isolation from each other, as well as mixtures of enantiomers, diastereomers and tautomers.

The term “salt thereof” characterises a pharmaceutically acceptable salt of cimetidine or a derivative of cimetidine in that a pharmaceutical acceptable salt may be substantially non-toxic and suitable for pharmaceutical use. A salt includes acid addition salt of cimetidine or its derivatives with an organic or inorganic acid. Illustrative examples of acid addition salts with inorganic acids are salts of cimetidine or a derivative thereof with bromide, chloride, dihydrochloride, hydrobromide, hydrochloride, iodide, nitrate, phosphate, sulfate or sulfonate or others known to those of ordinary skill in the art as the anion.

Illustrative examples of acid addition salts with organic acids are salts of cimetidine or a derivative thereof with acetate, adipate, ascorbate, benzenesulfonate, benzoate, besylate, bicarbonate, bitartrate, calcium edetate, camsylate, carbonate, citrate, edetate, edisylate, estolate, esylate, edisylate, estolate, formate, fumarate, gluceptate, gluconate, glucuronate, glutamate, glycollylarsanilate, hexylresorcinate, hippurate, hyclate, lactate, lactobionate, maleate, malate, mandelate, mesylate, methylbromide, methyinitrate, methylsulfate, mucate, napsylate, nitrate, pamoate, (embonate), pantothenate, phosphate/diphosphate, polygalacturonate, salicylate, stearate, subacetate, succinate, sulphate, sulfosalicylate, tannate, tartrate, teoclate, triethiodide, terephthalate, tosylate or triethiodide or others known to those of ordinary skill in the art as the anion.

Presently preferable embodiments include acid addition salts wherein the anion is selected from the group consisting of acetate, ascorbate, benzoate, citrate, fumarate, salicylate, sulphate, hydrochloride or phosphate. A presently preferred salt of cimetidine or a derivative is the hydrochloride salt.

Acid addition salts may be prepared from cimetidine or a derivative thereof because of the containment of a basic moiety by conventional chemical methods. By a basic moiety is meant an amino group that can be protonated, such as the secondary alkyl amino groups and the hetero-nitrogen of cimetidine. Generally, such addition salts may be prepared by reacting a free base of the cimetidine or a derivative thereof containing a free base with a stoichiometric amount of the appropriate acid in water or in an organic solvent, or in a mixture of the two. Generally, nonaqueous media like ether, ethyl acetate, ethanol, isopropanol, or acetonitrile are preferred. For example, appropriate acids include those derived from inorganic acids such as hydrochloric, hydrobromic, sulfuric acid, sulfamic acid, phosphoric acid, nitric acid, or the like; and those from organic acids such as mono and dicarboxylic aliphatic acids, phenylsubstituted alkanoic acids, hydroxyalkanoic acids, alkanedioic aicds, aromatic acids, e.g. acetic, propionic, succinic, glycolic, stearic, lactic, malic, tartaric, citric, ascorbic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, or the like.

In one embodiment the acid addition salts may be prepared with a fatty acid such as monocarboxylic acids including capric acid and lauric acid, or bile acids including glycocholic acid, glycodeoxycholic acid, cholic acid, deoxycholic acid, taurocholic acid and taurodexycholic acid so as to yield cimetidine caprate, cimetidine laurate, cimetidine glycodeoxycholate for example. For the preparation of fatty acid and bile acid addition salts see U.S. Pat. No. 6,255,502.

In further embodiments the acid addition salts may be prepared with metal carboxylic acid complexes such as Zn citrate so as to form N-methyl-N′-2-[(5-methylimidazol-4-yl)-methylthio]-ethyl}-N″-cyanoguanidine 2 hydroxy-1,2,3-propanetricarboxylate Zn²⁺ complex also known as cimetidine zinc citrate 1:1 complex. In other embodiments, citric acid can be replaced with tartaric acid or alkyl citric acid so as to form cimetidine zinc tartrate and cimetidine zinc alkyl citrates, respectively. The alkyl may be of any carbon length from 1 to 6, preferably from 1 to 4. In still further embodiments the complex may be solvated. For the preparation of addition salts with Zn carboxylic acids complexes, see U.S. Pat. No. 5,221,688. In other embodiments the metal carboxylic complex may be a bismuth carboxylic complex wherein the carboxylic acid is citric acid, tartaric acid, ethylenediaminetetraacetic acid, propylcitric acid or agaricic acid. In one particular embodiment, the acid addition salt is N-methyl-N′-2-[(5-methylimidazol-4-yl)-methylthio]-ethyl}-N″-cyanoguanidine 2 hydroxy-1,2,3-propanetricarboxylate bismuth³⁺ complex (cimetidine bismuth citrate) or solvates thereof. In another particular embodiment, the acid addition salt is cimetidine bismuth tartrate. For preparation of such addition salts with bismuth carboxylic acids complexes, see U.S. Pat. No. 5,273,984.

In further embodiments the salt is prepared with a metal salt of an organic or inorganic acid, e.g. zinc chloride and zink acetate dihydrate to form Zn-cimetidine compounds. Examples on Zn-cimetidine compounds are Zn₄(cimetidine)₆Cl₃(OH₅), Zn(cimetidine)Cl₂ and Zn₃(cimetidine)₄(CH3COO)₂(OH)₄. For the preparation of Zn-cimetidine compounds see U.S. Pat. No. 4,965,365.

As used herein, the term “prodrug” are considered to be any covalently bonded carriers which release the active parent drug (cimetidine or a derivative thereof) in vivo, in vitro or ex vivo. Preferably, the prodrug releases the drug in vivo when the prodrug is administered to a mammalian subject. Prodrugs of the compounds of the present invention are prepared by modifying functional groups present in the compounds in such a way that the modifications are cleaved, either in routine manipulation or in vivo, to yield the desired compound. As used herein prodrugs include compounds wherein the amino groups are bonded to any group that, when administered to a mammalian subject, is cleaved to form a free amino group, respectively. Examples of prodrugs include, but are not limited to, acetate, formate, or benzoate derivatives of the amine functional groups in the cimetidine or a derivative thereof.

In one embodiment, a prodrug of cimetidine may be in the form of a N-phosphoryloxymethyl prodrug (WO 99/33846).

As used herein, the term “metabolite” refers to the break-down or end product of cimetidine or a derivative thereof produced by metabolism or biotransformation in the animal or human body; e.g., biotransformation to a more polar molecule such as by oxidation, reduction, or hydrolysis, or to a conjugate. As used herein, the metabolite of cimetidine or a derivative thereof may be the therapeutically active form of the compound in the body.

As should also be understood, the term “cimetidine or derivatives thereof” preferably include derivatives that exhibit histamine H2 antagonism and/or anticancer activity. The histamine H2 antagonism may be such that the antagonism of a derivative may be from 0.1 to 100 times of the activity of cimetidine itself as determined in test methods for investigation of histamine H2-receptor antagonism. Preferably, the activity of a derivative relative to cimetidine should be in the range of from about 0.2 to 50, such as 0.2 to 40, such as 0.3 to 30, preferably from about 0.5 to 25 when the derivative and cimetidine is tested in the same molar concentration. The anticancer activity may be tested by the methods described in examples 3 or 4 as described infra. The anticancer activity of a derivative may be such that the anticancer activity in relation to cimetidine is in the range of from about 0.2 to 100, preferably in the range of from about 0.2 to 75, such as 0.3 to 60, such as 0.4 to 50, preferably from about 0.5 to 50 when the derivative and cimetidine is tested in the same molar concentration. The anticancer activity may be tested with and without concurrent presence of cysteine or a cysteine derivative.

Cysteine Derivatives

As used herein the term “cysteine derivative” is intended to mean cysteine or a derivative of cysteine according to formula I as well as mixtures thereof, stereoisomers thereof and/or salts thereof, wherein the hydrogen of the cysteine moiety may be replaced by a substituent. A derivative of cysteine is defined by the general Formula I:

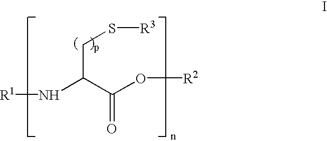

wherein n is an integer from 1 to 6;

p is a whole number selected from the group consisting of 0, 1 and 2;

R¹ is a monoradical selected from the group consisting of hydrogen, halogen, sulphate, optionally substituted C₁-C₈-acylene, optionally substituted C₁-C₈-alkylene, optionally substituted C₃-C₇-cycloalkylene, optionally substituted C₂-C₈-alkenylene, optionally substituted C₂-C₈-alkynylene. Furthermore, R¹ may be a monoradical selected from the group consisting of optionally substituted arylene, optionally substituted heterocyclylene, CH₂—N(R3′)(R3′), CH₂—OR3′, CH₂—SR3′, CH₂—O—C(═O)R3′, CH₂—O—C(═O)—OR3′, CH₂—O—C(═S)R3′, CH₂—S—C(═O)R3′, C(═O)(R3′), C(═S)R3′, —C(═S)—OR3′, —C(═O)—SR3′, C(═O)—N(R3′)(R3′), and C(C═S)—N(R3′)(R3′), wherein R3′ is selected from the group consisting of hydrogen, optionally substituted C₁-C₆-alkylene, optionally substituted C₂-C₆-alkenyl, optionally substituted C₂-C₆-alkynyl, optionally substituted aryl, optionally substituted heteroaryl, optionally substituted heterocycle, and optionally substituted C₃-C₇-cycloalkyl;

R² is a monoradical selected from the group consisting of hydrogen, halogen, sulphate, optionally substituted C₁-C₈-alkylene, optionally substituted C₃-C₇-cycloalkylene, optionally substituted C₂-C₈-alkenylene, optionally substituted C₂-C₈-alkynylene. Furthermore, R² may also be a monoradical selected from the group consisting of optionally substituted arylene, optionally substituted heterocyclylene, CH₂—N(R3′)(R3′), CH₂—OR3′, CH₂—SR3′, CH₂—O—C(═O)R3′, CH₂—O—C(═O)-OR3′, CH₂—O—C(═S)R3′, CH₂—S—C(═O)R3′, C(═O)(R3′), C(═S)R3′, —C(═S)—OR3′, —C(═O)—SR3′, C(═O)—N(R3′)(R3′), and C(C═S)—N(R3′)(R3′), wherein R3′ is selected from the group consisting of hydrogen, optionally substituted C₁-C₆-alkylene, optionally substituted C₂-C₆-alkenyl, optionally substituted C₂-C₆-alkynyl, optionally substituted aryl, optionally substituted heteroaryl, optionally substituted heterocycle, and optionally substituted C₃-C₇-cycloalkyl; and

R³ is a monoradical selected from the group consisting of hydrogen, sulphate, optionally substituted C₁-C₈-alkylene, optionally substituted C₁-C₈-alkenylene, optionally substituted C₂-C₈-alkynylene, optionally substituted arylene, optionally substituted heteroarylene and

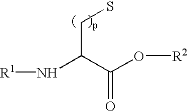

wherein p, R¹ and R² are independently selected from their groups as defined above.

The term “optionally substituted” and the term “hydrogen replacement” is intended to mean the substitution of one or more hydrogen atoms, which is substituted with another atom, chemical group or entity, termed substituents. Illustrative examples of substituents include carboxyl, formyl, amino, hydroxyl, halogen, nitro, sulphono, sulphanyl, C₁₋₆-alkyl, aryl, aryloxy, aryloxycarbonyl, arylcarbonyl, heteroaryl, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, C₁₋₆-alkoxyl, oxo, C₁₋₆-carboxyl, C₁₋₆-alkoxycarbonyl, C₁₋₆-alkylcarbonyl, wherein said aryl and heteroaryl are intended to denote an aryl and a heteroaryl, respectively, that are substituted 1-3 times with C₁₋₆-alkyl, C₁₋₆-alkoxy, nitro, cyano, hydroxy, amino or halogen. Preferably, the hydrogen is substituted with halogen, nitro, cyano, C₁₋₆-alkyl and/or aryl, wherein the aryl preferably is substituted once or twice with C₁₋₆-alkyl, nitro, cyano, hydroxy, amino or halogen.

The term “wherein the hydrogen of the cysteine moiety may be replaced by a substituent” is intended to characterise the substitution of the amino-hydrogen present in formula I, with another atom, chemical group or entity selected from the group consisting of halogen, nitro, cyano, sulphono, sulphanyl, C₁₋₆-alkyl, aryl, heteroaryl, wherein said aryl and heteroaryl are intended to denote an aryl and a heteroaryl, respectively, that are substituted 1-3 times with C₁₋₆-alkyl, C₁₋₆-alkoxy, nitro, cyano, hydroxy, amino or halogen. Preferably, the substituent is selected from the group consisting of halogen, nitro, cyano,

C₁₋₆-alkyl and/or aryl, wherein the aryl preferably is substituted once or twice with C₁₋₆-alkyl, nitro, cyano, hydroxy, amino or halogen.

The term “C₁-C₈ alkylene” is intended to mean a linear or branched saturated hydrocarbon chain wherein the longest chain has from one to eight carbon atoms, such as methyl, ethyl, n-propyl, isopropyl, n-butyl, isobutyl, sec-butyl, tert-butyl, pentyl, isopentyl, neopentyl, hexyl, heptyl, octyl chain. The C₁-C₈ alkylene chain may optionally be substituted. In presently interesting embodiments, the C₁-C₈ alkylene chain of R¹, R², R³ is has its longest alkylene chain from one to 5 carbon atoms; C₁-C₅ alkylene.

Likewise, the term “C₂-C₈-alkenylene” is intended to mean a linear or branched unsaturated hydrocarbon chain with one or more double bonding(s) wherein the longest chain has from two to eight carbon atoms. The C₂-C₈ alkenylene chain may optionally be substituted. In presently interesting embodiments, the C₂-C₈ alkenylene chain of R¹, R², R³ has the longest alkenylene chain from one to 5 carbon atoms, C₂-C₅ alkenylene.

The term “C₂-C₈-alkynylene” is intended to mean a linear or branched unsaturated hydrocarbon chain with one or more triple bonding(s) wherein the longest chain has from two to eight carbon atoms. The C₂-C₈ alkynylene chain may optionally be substituted. In presently interesting embodiments, the C₂-C₈ alkynylene chain of R¹, R², R³ has the longest alkynylene chain from one to 5 carbon atoms, C₂-C₅ alkynylene.

The term “halogen” includes fluorine, chlorine, bromine and iodine.

The term “C₁-C₈-acylene” characterises an alkylene that is linear or branched, saturated or unsaturated, preferably monounsaturated, or an arylene, wherein said alkylene and arylene has a —C═O group. Non-limiting examples are formyl, acetyl and benzoyl. The C₁-C₈-acylene may optionally be substituted with an amino, a cyano, a hydroxy, a halogen, a nitro, sulphono, sulphonyl and/or a thiol group.

The term “arylene” characterises a phenyl ring optionally substituted 1-3 times with C₁-C₆-alkylene, amino, carboxyl, cyano, hydroxy, halogen, nitro, thiol group, wherein the C₁-C₆-alkylene is optionally substituted with amino, a cyano, a hydroxy, a halogen, a sulphono, a sulphonyl and/or a thiol group. Preferably, the arylene is monosubstituted or substituted twice.

In presently preferred embodiments of the invention, the cysteine derivatives is defined by Formula I, wherein n is an integer from 1 to 3; and p is a whole number selected from the group consisting of 0, 1 and 2. In further interesting embodiments thereof, R¹ is a monoradical selected from the group consisting of hydrogen, halogen, sulphate, optionally substituted C₁-C₈-acylene, optionally substituted C₁-C₈-alkylene, optionally substituted C₃-C₇-cycloalkylene, optionally substituted C₂-C₈-alkenylene and/or optionally substituted arylene. Preferably, R¹ is a monoradical selected from the group consisting of hydrogen and optionally substituted C₁-C₈-acylene.

In some embodiments or still further embodiments, R² is a monoradical selected from the group consisting of hydrogen, halogen, optionally substituted C₁-C₈-alkylene, optionally substituted C₃-C₇-cycloalkylene, optionally substituted C₂-C₈-alkenylene and/or optionally substituted arylene. In presently interesting embodiments, R² is a monoradical selected from the group consisting of hydrogen, halogen, optionally substituted C₁-C₈-alkylene, alkenylene and/or optionally substituted arylene, preferably R² is a monoradical selected from the group consisting of hydrogen, halogen and/or optionally substituted C₁-C₈-alkylene.

In some embodiments or still further embodiments, R³ is a monoradical selected from the group consisting of hydrogen, sulphate, optionally substituted C₁-C₈-alkylene, optionally substituted C₃-C₇-cycloalkylene, optionally substituted C₂-C₈-alkenylene and/or optionally substituted arylene. Preferably, R³ is a monoradical selected from the group consisting of hydrogen, sulphate, optionally substituted C₁-C₆-alkylene, optionally substituted acylene and optionally substituted arylene. Most preferably, R³ is selected from the group consisting of hydrogen and sulphate.

In particular embodiments of the invention, the cysteine derivative is defined by Formula I, wherein n is selected from the group consisting of 2 and 3; and R³ is a monoradical selected from the group consisting of hydrogen, sulphate, optionally substituted C₁-C₈-alkylene, optionally substituted C₁-C₈-alkenylene, optionally substituted C₂-C₈-alkynylene, optionally substituted arylene and/or optionally substituted heteroarylene. Preferably, R³ is a monoradical selected from the group consisting of hydrogen, sulphate, optionally substituted C₁-C₆-alkylene and/or optionally substituted arylene. Most preferably, R³ is selected from the group consisting of hydrogen and sulphate.

In another particular embodiment of the invention, the cysteine derivative is defined by Formula I, wherein n is 1 and R³ is

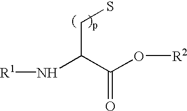

It should further be understood that the chain length of each of the substituents may be shorter. For example, C₁-C₈ alkylene may be from C₁-C₆ alkylene, such as C₁-C₅ alkylene, preferably C₁-C₄ alkylene such as C₁-C₃ alkylene. Likewise, the C₂-C₈-alkenylene and C₂-C₈-alkynylene may be from C₂-C₆ alkenylene/alkynylene, such as C₂-C₅ alkenylene/alkynylene, preferably C₂-C₄ alkenylene/alkynylene such as C₂-C₃ alkenylene/alkynylene. Furthermore, the acylene group may comprise fewer carbon atoms such as C₂-C₆ acylene, such as C₂-C₅ acylene, preferably C₂-C₄ acylene such as C₂-C₃ acylene. The C₃-C₇-cycloalkylene may be C₃-C₆-cycloalkylene, such as C₃-C₅-cycloalkylene.

Without being limited to a particular theory, it is the current understanding that the thiol group of the cysteine moiety is important for the activity of the complex. Thus, typically, the thiol group is not derivatised, i.e. R³S is hydrogen. As the person skilled in the art, even within the current theory would know it, R³ may be such that upon administration, the in vivo hydrolysis of R³S would provide the free thiol SH.

Moreover, the chemical complexes and compositions of the present invention may comprise cysteine derivative precursors, which upon administration and in vivo chemical modification or enzymatic modification provide a derivative of cysteine according to Formula I. Thus, in one embodiment, the chemical complexes and compositions comprise N-acetylated cysteine or a N-acetylated cysteine derivative that are deacetylated in vivo to form cysteine and a cysteine derivative, respectively.

Suitable embodiments of cysteine derivatives of formula I may be the N-acetyl derivative, as discussed supra, but also be other cysteine derivatives such as the free amine (NH₂, wherein R¹ is a hydrogen), the N-benzyl, N-benzoyl, other N-acyl derivatives and N-alkyl derivatives. An embodiment, wherein R¹ results in a prodrug such that the free amine is generated in vivo is a particularly interesting aspect of the invention. Cysteic acid and cystine are alternative putative sources of cysteine in vivo. Similarly, the free amine or quaternary ammonium salts of the amine of compounds of formula II are interesting embodiments of compounds of formula II, such as cysteine hydrochloride.

Suitable embodiments of compounds of formula I are such that R³ is HOOC—CH₂—S, as in carboxymethyl cysteine (carbocysteine). In a further embodiment of the invention, the cysteine derivative may consist of homocysteine.

The term “cysteine derivative” is furthermore intended to mean cysteine dimers, oligomers, and polymers, wherein up to six cysteine moieties are included, such as peptides of cysteine, wherein the N-terminal end is preferably acetylated. In one embodiment, the cysteine derivative is glutathione or N-acetylated derivative thereof.

In presently preferred embodiments of the invention, the cysteine derivative(s) of Formula I is N-acetylcysteine. N-acetylcysteine may be obtained from natural sources or synthetically. However, N-acetylcysteine may also be obtained from precursors, which upon chemical or enzymatic reaction release free N-acetylcysteine. Such chemical or enzymatic release from precursors of N-acetylcysteine may take place either in vivo after administering the precursor or outside the body. A particularly suitable example of a potential precursor is cysteine itself, which may be acetylated by bacteria in the gut lumen or enzymatically during the penetration of the gut wall into the systemic circulation. Cysteine may be acetylated to N-acetylcysteine in a pharmaceutical formulation containing acetylating bacteria, e.g. E. coli bacteria and lactic bacteria.

In typical embodiments of the invention, the cysteine derivative(s) of Formula I is cysteine, N-acetylcysteine, cystine, homocysteine, cysteine methylester, S-ethyl-cysteine, N,S-isobuturyl-cysteine, S-carboxymethyl-cysteine, S-ethyl-homocysteine, S-methyl-cysteine, cysteine S-sulfate, N,S-diacetyl-cysteine methylester, N-acetyl-S-methylcysteine, glutathione, stereoisomers thereof, salts thereof and/or mixtures thereof. In presently suitable embodiments of the invention, the cysteine derivative is selected from the group consisting of cysteine, cystine, N-acetylcysteine, homocysteine, glutathione, salts thereof and/or mixtures thereof, more preferably selected from the group comprising of cystine, N-acetylcysteine, glutathione, salts thereof and/or mixtures thereof.

Furthermore, the term “cysteine derivative” is denoted to mean a salt of cysteine as well as a salt of a cysteine derivative, Thus, it should also be understood that salts of compounds of formula 1 are anticipated, including for instance hydrates, solvent addition forms, base addition salts or acid addition salts. In the event where the cysteine or a cysteine derivative has a free carboxylic acid group base addition salts are anticipated. The term “base addition salts” include alkali metals, such as sodium and potassium, alkali earth metals, such as calcium and magnesium, and organic addition salts such as quaternary ammonium cations of cysteine or a derivative thereof. Hence, in present interesting embodiments, the base addition salt of cysteine or a cysteine derivative includes a cation selected from the group consisting of Na⁺; K⁺; Mg⁺⁺; Ca⁺⁺ or NH₄⁺.

The term “acid addition salts” is denoted to mean acid addition salt of cysteine derivatives with a basic moiety, such as a free amino group, with an organic or inorganic acid. Suitable acid addition salts is selected from the group consisting of hydrochloride, citrate, ascorbate, acetyl, formyl and/or benzoyl salts of a cysteine derivative.

Finally, the present inventor anticipates prodrug derivatives of the cysteine derivative. The prodrug form may be the result of the derivatisation of the amino group or another functional group present on the cysteine derivative, as is known to the person skilled in the art.

Chemical Complexes, Compositions and Therapeutic Use

In one embodiment, the composition comprises the cysteine derivative of Formula I and cimetidine or a derivative thereof of Formula II in the form of a chemical complex together with a pharmaceutically acceptable carrier and/or excipient. In another embodiment, the composition only contains said complex, optionally the said complex comprises solvent residues.

The term “chemical complex” is intended to include the definition defined by IUPAC that read as follows:

“A molecular entity formed by loose association involving two or more component molecular entities (ionic or uncharged), or the corresponding chemical species. The bonding between the components is normally weaker than in a covalent bond.” (IUPAC Compendium of Chemical Terminology 2nd Edition (1997))

Thus, the term “chemical complex” is intended to mean any combination of the component molecules that does not imply a covalent bonding between the component molecules. Also as used herein, the chemical complex of the present invention relates to a complex obtainable from the combining of a cysteine derivative of Formula I and cimetidine or a derivative thereof of Formula II.

The complexes of the invention may be prepared according to a number of different methods, which are obvious to a person skilled in the art. The following procedures are non-limiting examples of such methods:

The components of the complex, dosed in appropriate amounts to give the correct molar ratio between the moieties, are dissolved, dispersed, or suspended in an appropriate solvent, for example water, an organic solvent or mixtures thereof. Non-limiting examples of suitable organic solvents are ethanol, methanol, iso-propyl alcohol, acetone, hexane, ethylacetate or mixtures thereof. The solvent is then removed by a technique suitable for the complex, for example but not limited to evaporation, in vacou evaporation, spray drying, freeze-drying, fluid bed drying or spin flash drying. Alternatively the complex may be obtained by precipitation and subsequent centrifugation or filtering.

As stated the combination of the two kinds of compounds provides a surprisingly effective therapeutic agent for inhibiting tumor growth. The proper therapeutic efficacy may, in part, be adjusted by providing the two agents in suitable molar ratios or mass ratios.

The molar ratio between cimetidine or a derivative thereof and the cysteine derivative may be about 1:10000 to 10000:1, such as 1:1000 to 1000:1 preferably about 1:500 to 500:1, such as 1:100 to 100:1, about 1:50 to 50:1, or about 1:40 to 40:1, also about 1:30 to 30:1, such as about 1:25 to 25:1, about 1:20 to 20:1, about 1:18 to 18:1, about 1:16 to 16:1, about 1:14 to 14:1, or about 1:12 to 1:12, also about 1:10 to 10:1, such as about 1:9 to 9:1, about 1:8 to 8:1, about 1:7 to 7:1, about 1:6 to 6:1, also from 1:5 to 5:1 such as from 1:4 to 4:1, e.g. from 1:3 to 3:1, such as from 1:2 to 2:1.

Alternatively defined, the ratio between cimetidine or a derivative thereof and the cysteine derivative may be expressed as a mass ratio. The mass ratio between cimetidine or a derivative thereof and the cysteine derivative may be about 1:10000 to 10000:1, such as 1:1000 to 1000:1 preferably about 1:500 to 500: 1, such as 1:100 to 100:1, about 1:50 to 50:1, or about 1:40 to 40:1, also about 1:30 to 30:1, such as about 1:25 to 25:1, about 1:20 to 20:1, about 1:18 to 18:1, about 1:16 to 16:1, about 1:14 to 14:1, or about 1:12 to 1:12, also about 1:10 to 10:1, such as about 1:9 to 9:1, about 1:8 to 8:1, about 1:7 to 7:1, about 1:6 to 6:1, also from 1:5 to 5:1, such as from 1:4 to 4:1, e.g. from 1:3 to 3:1, such as from 1:2 to 2:1.

It should further be understood that according to the invention the chemical complexes and/or compositions further comprising one or more therapeutically active agents in order to strengthen, improve, potentiate, or prolong the therapeutic actions of said complexes and said compositions. Of particular relevance is wherein the therapeutically active agent is an anticancer drug, such as therapeutically active agents that are selected from the group consisting of DNA-interactive agents, antimetabolites, tubulin-interactive agents and/or hormonal agents. Typical examples of DNA-interactive agents, antimetabolites, tubulin-interactive agents and hormonal agents are listed below. In presently interesting embodiments, the further therapeutically active agents include DNA-interactive agents and/or tubulin interactive agents. Other interesting agents include protease inhibitors, cyclooxygenase inhibitors, nuclear factor kappa B inhibitors, 3-hydroxy-3-methylglutaryl-coenzyme A (HMG-CoA) inhibitors, vitamin D derivatives, vitamin D analogs. antioxidants, and/or agents that improve the immune response such as agents that affect the TH-1 arm the immune system, such as interleukin 2 and/or 12. In a presently preferred embodiment, the further anticancer agent is capecitabine (Xeloda®). A list of further anticancer agents is described infra.

However, in some embodiments the chemical complex and/or the composition comprise as the only therapeutically active substances cimetidine or a derivative thereof and a cysteine derivative. In particularly, wherein the cysteine derivative is cysteine the composition does not contain a leukotriene antagonist, in particular does not contain the agent montelukast. Furthermore, p-aminobenzoic acid and derivatives thereof may be unwanted for use in the compositions of the invention. Thus, some embodiments do not comprise p-aminobenzoic acid or derivatives thereof.

For the administration to a mammal, such as a human, the chemical complex may be administered directly, eventually provided in a capsule or the like. More convenient, the complex may be formulated into a composition comprising the chemical complex and optionally, one or more acceptable excipients. Alternatively, the combination of the two agents may also be formulated into a composition without being provided as a chemical complex. Thus, in some embodiments of the invention, the chemical complexes or compositions further comprise one of more excipient(s) or carrier(s), preferably pharmaceutically acceptable excipient(s) or carrier(s).

The compositions according to the present invention may be formulated as a pharmaceutical composition for peroral, oral, topical, trans-mucosal, trans-dermal or parenteral administration or formulated as an implant, preferably the composition is formulated for peroral or topical administration.

In the present invention the term “pharmaceutical composition” relates to a composition that is formulated or pre-formulated for administration by peroral, oral, topical, trans-mucosal, trans-dermal or parenteral means. In the event, where the composition is preformulated the composition may be ready to administration upon admixing it with further expients or carriers.

The term “peroral administration” is intended to mean administration to an individual of a composition trough the mouth, preferably where the release and absorption of the therapeutically active agent is not intended to occur in the oral cavity, but rather after passing the oral cavity, such as in the gastrointestinal tract.

The term “oral administration” is intended to denote the administration of a composition to the oral cavity for release and absorption of the therapeutically active agent in a defined part of the mouth, e.g. buccal, sublingual, or gingival.

The term “topical administration” characterise adminstration to a restricted circumscribed area, the area in this embodiment are directed to skin or mucosal membranes. As used herein the terms “mucosal” or “mucosa” relate to, but not limited to, the epithelial membranes lining the oral, nasal, rectal, vaginal and ocular cavities. Thus, topical administration is selected from the group consisting of administration to the mucosa of the oral cavity, nasal mucosa, rectal mucosa, vaginal mucosa and/or ocular mucosa

Moreover, the term “topical administration” is not intended to be limited to topical application of a composition directly on the circumscribed area. In some embodiments the delivery of the therapeutically active agent, or a part thereof, can occur by other means, e.g. by peroral administration upon where the therapeutically active agent is directed to the colon for being delivered to the desired target area of the colon without being substantially absorbed therefrom. That is to say where the agent should exert its therapeutically effect locally in the GI tract, preferably in the colon.

The term “trans-dermal administration” characterises administration of an agent/a composition to an individual through derma or any skin surface. Likewise “trans-mucosal administration” characterise administration of an agent/a composition trough musosa. The administration includes application of solid, semisolid or liquid formulations for local or systemic action. By the term “skin” or “dermal” is meant any skin surface.

The term “parenteral administration” characterises administration by some means other than the alimentary channel, specifically administration to a muscle, vein, or any other pathway other than the mouth, skin or mucous to introduce a composition into an individual.

By the term “implants” are meant long acting formulations administered by implantation (for example subcutaneously or intramuscularly) or by intramuscular injection.

In a suitable embodiment of the invention, the compositions are administered by peroral administration. In another suitable embodiment of the invention the compositions are used for topical administration. Furthermore, in interesting embodiments of the invention, the composition is delivered to the colon e.g. by formulation of said composition for delivery of the therapeutically active agents to the gastrointestinal tract such as formulated for delivery to colon.

Cimetidine or a derivative thereof and the cysteine derivative may together be comprised in a single formulation or may each individually be comprised in separate formulations. The separate formulations may be administered in a simultaneous or non-simultaneous manner. As stated, cimetidine or a derivative thereof and the cysteine derivative are together comprised in a single formulation, such as a dosage unit

The term “dosage unit” relates to a composition formulated in a unit comprising one or more daily doses, wherein the unit preferably releases one to four daily dose per 24 hours.

The magnitude of a prophylactic or therapeutic dose of an active ingredient in the acute or chronic management of a disorder or condition will vary with the severity of the disorder or condition to be treated and the route of administration. The term “dose” relates to an amount of given at one time of a therapeutic drug or a diagnostic agent. The dose, and perhaps the dose frequency, will also vary according to age, body weight, response, and the past medical history of the patient. Suitable dosing regimens can be readily selected by those skilled in the art with due consideration of such factors.

The active ingredients of the chemical complex or pharmaceutical composition of the present invention need not be administered as one pharmaceutical entity, but may of course be administered as individual compounds or pharmaceutical compositions. In addition to the formulations described previously, the compositions of the invention may also be formulated as a depot preparation.

The pharmaceutical compositions for peroral, oral, topical, transdermal, or parenteral administration may be in form of, e.g., solid, semi-solid or fluid compositions such as a suspension and an emulsion, and formulated according to conventional pharmaceutical practice, see, e.g., “Remington: The science and practice of pharmacy” 20^{th} ed. Mack Publishing, Easton Pa., 2000 ISBN 0-912734-04-3 and “Encyclopedia of Pharmaceutical Technology”, edited by Swarbrick, 3. & J. C. Boylan, Marcel Dekker, Inc., New York, 1988 ISBN 0-8247-2800-9.

The choice of pharmaceutically acceptable excipients in a composition for use according to the invention and the optimum content thereof is determined on the basis of the selection of cimetidine or a derivative thereof, selection of the cysteine derivative, the kind of dosage form chosen and the mode of administration.

A pharmaceutically acceptable excipient is a substance, which is substantially harmless to the individual to which the composition will be administered. Such an excipient suitably fulfils the requirements given by the national drug agencies. Official pharmacopeias such as the British Pharmacopeia, the United States of America Pharmacopeia and the European Pharmacopeia set standards for well-known pharmaceutically acceptable excipients.

The peroral compositions for use according to the invention include an array of solid, semi-solid and fluid compositions.

Solid peroral dosage forms of the invention that are suitable for peroral administration can be presented as discrete dosage forms. Compositions of particular relevance are e.g. solutions, suspensions, emulsions, uncoated tablets, immediate-release tablets, modified-release tablets, gastro-resistant tablets, orodispersible tablets, efferverscent tablets, chewable tablets, soft capsules, hard capsules, modified-release capsules, gastro-resistant capsules, uncoated granules, effervescent granules, granules for the preparation of liquids for peroral use, coated granules, gastro-resistant granules, modified-release granules, powders for peroral administration and powders for the preparation of liquids for peroral use.

Typical peroral dosage forms of the invention are prepared by combining the active ingredient(s) in an intimate admixture with at least one excipient according to conventional pharmaceutical compounding techniques.

If desired, the compositions can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Suitable coating agents may be selected from the group comprising hydroxypropylcellulose, hydroxypropylmethylcellulose, polyvinylpropylidone, ethylcellulose and polymethylacrylates. Furthermore, in suitable embodiments of the invention, the compositions are enteric coated so as avoid release of the active agents in the stomach.

In a further suitable embodiment of the invention, the composition is formulated as a modified release dosage form. Modified release dosage forms include, without limitation dosage forms with immediate release, extended release, pulse release, variable release, controlled release, timed release, sustained release, delayed release, long acting, and combinations thereof.

The topical, trans-mucosal and trans-dermal compositions for use according to the invention include, but are not limited to, an array of solid, semi-solid and fluid compositions. Compositions of particular relevance are e.g. pastes, ointments, hydrophilic ointments, hydrophobic ointments, water-emulsifying ointments, creams, gels, hydrogels, pastes, solutions, emulsions, suspensions, lotions, liniments, resoriblets, suppositories, enema, pessaries, moulded pessaries, vaginal capsules, vaginal tablets, shampoos, jellies, soaps, sticks, sprays, powders, films, foams, pads, sponges (e.g. collagen sponges), pads, tampons, dressings (such as, e.g., absorbent wound dressings), drenches, bandages, plasters and transdermal delivery systems or other forms known to one of skill in the art.

Further aspects of the invention relate to the therapeutic effects observed for the chemical complexes and the compositions of the invention. As mentioned, it was surprisingly found that the combination of N-acetyl-cysteine and cimetidine in the form of a chemical complex of the invention exhibited effective anti-cancer activity.

Thus, in a broadly sense the chemical complexes or compositions of the invention provides an anti-cancer effect. Given the therapeutic actions of the combination of a cysteine derivative of Formula I and cimetidine or a derivative thereof of Formula II, the use of a combination of a cysteine derivative and cimetidine or a derivative thereof, for example in the form of a composition or in the form of a chemical complex, for the preparation of a medicament for the treatment of cancer and/or chemoprevention in a mammal is a further aspect of the invention. Also a related aspect relates to the use of a combination of a cysteine derivative of Formula I and cimetidine or a derivative thereof, for example in the form of a composition or in the form of a chemical complex, for the preparation of a medicament for the immunomodulation of a mammal.

Moreover, a still further aspect relates to a method for treating cancer in a mammal, comprising administration to said mammal of an effective amount of a combination of cimetidine or a derivative thereof and a cysteine derivative, or pharmaceutically acceptable salts thereof, or a chemical complex comprising said combination or said salts to said mammal. Furthermore, a still further aspect relates to a method for chemoprevention in a mammal, comprising administration to said mammal of an effective amount of a combination of cimetidine or a derivative thereof and a cysteine derivative, or pharmaceutically acceptable salts thereof, or a chemical complex comprising said combination or said salts to said mammal.

A related aspect relates to a method for immunomodulation in a mammal, comprising administration to said mammal of an effective amount of a combination of cimetidine or a derivative thereof and a cysteine derivative, or pharmaceutically acceptable salts thereof, or a chemical complex comprising said combination or said salts to said mammal.

As defined herein, the term “mammal” is intended to include a patient, animal, or a human, who will benefit from the method of this invention. This patient, animal or human may be a person genetically disposed to cancer or a patient, animal or human who is believed to be at risk for developing cancer or is diagnosed with cancer.

As used herein, the term “effective amount” relates to the effective dose to be determined by a qualified practitioner, who may titrate dosages to achieve the desired response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration, or other factors known to a medical practitioner.

The term “anti-cancer” activity is denoted to mean any type of inhibition of cancer cells or tumors, e.g. by disrupting cancer cell proliferation, inhibiting tumor-related angiogenesis or preventing cancer cells from metastasising by inhibition of adhesion molecules such as E-selectin, inducing cancer specific apoptosis or by cancer chemoprevention. Anticancer activity may also imply promotion of host resistance to cancer, e.g. stimulation of cancer-specific immune responses. The anti-cancer activity may be related to the reducing effect of a therapeutic agent on the growth of cancer cells of various origins, such as wherein the growth is inhibited by more than 10%, such as more than 20%, such as more than 30%, such as more than 40, 50, 75, 100, 12, 150 or more than 200% in comparison to control within 5 days such as within 10 days of the start of the test. Such test methods are known to the skilled person, or may be carried out according to examples 3 or 4 with the exception that the test may include other cancer cells than of colonic origin, such as of epithelial origin or of mesenchymal origin.

As used herein, the term “treatment” relates to treatment of symptoms or prevention of the relapse of symptoms in a person diagnosed with cancer. Given, that the combination of agents according to the present invention is effective in reducing inflammatory reactions, the term “treatment” also relates to treatment of symptoms or prevention of the relapse of symptoms in a person diagnosed with a disease related to inflammation, hypersensitivity, infection, and pain.

It is contemplated that the chemical complexes and compositions of the invention may have particular relevance for the treatment of cancers of the gastrointestinal system selected from the group consisting of colon cancer, rectal cancer, colorectal cancer, pancreatic cancer, stomach (gastric) cancer, esophegeal cancer, liver cancer and/or bladder cancer. Furthermore, the complexes and compositions of the invention may have a therapeutic potential in metatstatic as well as invasive cancers selected from the group consisting of breast cancer, cancer of the male and female genital tract, cancer of the thymus, lung, stomach, small intestine, prostate, adrenal gland, pancreas, colon, lymphoid tissue, liver, brain, salivary gland, spleen and/or skin.

Thus, in one embodiment of the invention, the combination of a cysteine derivative and cimetidine or a derivative thereof is for the treatment of cancer selected from the group of cancer in the gastrointestinal system, metastatic cancers and/or invasive cancers. It should be understood that the complexes and compositions of the invention are relevant for the treatment of a broad range of cancers related to gastrointestinal system, metastatic cancers and/or invasive cancers. The following are non-limiting examples:

1) Carcinomas including acinar cell carcinoma, adenoid cystic carcinoma, carcinoma of adrenal cortex, carcinoma of ampulla of vateri, anaplastic carcinoma of nasopharynx, anaplastic carcinoma of thyroid, basal cell carcinoma, carcinoma of bile ducts, bronchoalveolar carcinoma, bronchogenic carcinoma, carcinoma of C cells, carcinoma of clear cells of the vagina, colorectal carcinoma, follicular carcinoma of the thyroid, carcinoma of the gallbladder, carcinoma of the kidney, hepatocellular carcinoma, lymphangitic carcinoma of the lung, medullary carcinoma of the breast, medulluary carcinoma of the thyroid, mucoepidermoid carcinoma of the salivary glands, nasopharyngeal carcinoma, carcinoma of the pancreas, papillary carcinoma of the thyroid, carcinoma of the parathyroid, renal cell carcinoma, scirrhous carcinoma of the breast, secretory carcinoma of the endometrium, small cell carcinoma of the lung, squamous cell carcinoma, carcinoma of the stomach, carcinoma of thy thymus, transitional cell carcinoma of the bladder, verrucous carcinoma of the penis, verroucous carcinoma of the vulva, carcinoma in situ of the bladder, carcinoma in situ of the breast and carcinoma in situ of the penis.

2) Sarcomas including alveolar rhabodmyosarcoma, sarcoma of adipose tissue (liposarcoma), sarcoma of bone i.e. in paget disease, angiosarcoma, chondrosarcoma, sarcoma botryoides, cystocarcoma phyllodes, embryonal rhabdomyosarcoma, Ewing sarcoma, fibrosarcoma, granulocytic sarcoma, hemangiosarcoma, Kaposi's sarcoma, leiomyosarcoma, malignant fibrous histicytoma, neurofibrosarcoma, ostegenic sarcoma, osteosarcoma, rhabdomosarcom, synovial sarcoma and sarcoma botyroides of the vagina.

3) Hepatoma of the liver, malignant melanoma, leukaemia, lymphoma, seminoma of the testis, astrocytoma, glioblastoma multiforme, oligodendroglioma, ependymoma, medulloblastoma, meningioma, retinoblastoma, neuroblastoma, plasma cell neoplasia.

Additionally, the combination of a cysteine derivative and cimetidine or a derivative thereof may also have other immunomodulating activities, such as suppression of hypersensitivity reactions, suppression of inflammatory reactions, suppression of cartilage degeneration, suppression of IgE mediated allergic reactions, suppression of autoimmune reactions, and/or reduction of pain.

Therefore, in further embodiments of the invention, the immunomodulating activity relates to the suppression of inflammatory reactions such as treatment of diseases and disorders, or symptoms associated therewith, selected from the group consisting of hypersensitivity skin disease, atopic eczema, contact dermatitis, seborrhoeic eczema, psoriasis, IgE mediated allergic reactions, asthma, allergic rhinitis, anaphylaxis, autoimmune disease, chronic inflammatory disease, Crohn's disease, ulcerative colitis, rheumatoid arthritis, gout, osteoarthritis and pain.

The manner of combining the cysteine derivative(s) and cimetidine or a derivative thereof in a medicament for treating cancer and/or exhibiting other immunomodulating activities may be done in an array of manners of administration. The cysteine derivative and cimetidine or a derivative thereof may together be comprised in a single formulation or may each individually be comprised in separate formulations such as separate dosage units.

Furthermore, the manner of administration may be such that said combination is administered in a simultaneous or non-simultaneous manner. Thus, a cysteine derivative of Formula I may be administered first and cimetidine or a derivative thereof may be administered simultaneously or subsequently, or in an opposite order of administration.

However, in a preferred embodiment, the cysteine derivative(s) of Formula I and the cimetidine or derivative thereof are administered simultaneously, preferably in the form of a chemical complex as defined supra. As stated, the cysteine derivative(s) of Formula I and the cimetidine or derivative thereof are administered simultaneously in the form of a pharmaceutical composition as defined supra in a single formulation, such as a single dosage unit.

It should also be understood that the combination of a cysteine derivative and cimetidine or a derivative thereof may be administered daily, even for a longer period such a 0.5, 1, 2, 3, 4, 5, or 10 years, in that the combination is substantially free of adverse effects. This is not the case with other anticancer agents such as the cyclooxygenase inhibitors.

According to the use of a cysteine derivative of Formula I and cimetidine or a derivative thereof for the preparation of a medicament and to methods for treating cancer, providing chemoprevention or exhibiting immunomodulation, the product may further comprise one or more therapeutically active agents.

Methods and uses as defined herein as well as chemical complexes and compositions defined herein may additionally be combined with other anticancer agents to provide an operative combination for improving the cancer treatment. Preferably, the further anticancer agent may be active in the treatment of cancers of the gastrointestinal system selected from the group consisting of colon cancer, rectal cancer, colorectal cancer, pancreatic cancer, stomach (gastric) cancer, esophegeal cancer, liver cancer and/or bladder cancer. The combination with a further anticancer agent is intended to include any chemically compatible combination of anticancer agents with the chemical complexes and/or compositions of the present invention, as long as the combination does not eliminate the anticancer activity of the additional agent or the activity of the combination of cimetidine or a derivative thereof and the cysteine derivate of the present invention. For example, the chemical complex or compositions of the invention can be combined with other anticancer agents, chemotherapeutic agents, potentiators or pharmaceutically acceptable derivatives or salts thereof.

Thus, according to the invention, the uses and methods as described further comprise one or more therapeutically active agent, preferably wherein the therapeutically active agent is an anticancer agent that is preferably selected from the group consisting of DNA-interactive agents, antimetabolites, tubulin-interactive agents and/or hormonal agents. Other interesting agents include protease inhibitors, cyclooxygenase inhibitors, nuclear factor kappa B inhibitors, 3-hydroxy-3-methylglutaryl-coenzyme A (HMG-CoA) inhibitors, vitamin D derivatives and vitamin D analogs, antioxidants, and/or agents that improve the immune response such as agents that affect the TH-1 arm the immune system, such as interleukin 2 and/or 12.

The combination therapy can be sequential, that is the treatment with one agent first and then the second agent, or it can be treatment with both agents at the same time. The sequential therapy can be within a reasonable time after the completion of the first therapy before beginning the second therapy. The treatment with both agents at the same time can be in the same daily dose or in separate doses. For example treatment with one agent on day 1 and the other on day 2. The exact regimen will depend on the disease being treated, the severity of the disease and the response to the treatment. As used herein, “adjunct therapy” means that the patient in need of the drug is treated or given another drug for the disease and/or a potentiator in conjunction with the chemical complexes and compositions of the invention. Adjunct therapy can be sequential therapy where the patient is treated first with one agent and then the other within a given time period or concomitant therapy where the two agents are administered substantially simultaneously or in overlapping dosing regimens.

“Potentiators” are materials, which affect the body's response or diseased cell's response to an agent. A “potentiator” can be any material, which improves or increases the efficacy of a pharmaceutical composition or acts as an immunomodulator to increase the efficacy of an agent.

The anticancer agents, which can be used in adjunct therapy or as further therapeutically active agents with cimetidine or a derivative thereof and the cysteine derivate in compositions or chemical complexes of the invention are generally classified as DNA-interactive agents, antimetabolites, tubulin-interactive agents, hormonal agents and others such as asparaginase or hydroxyurea. Each of the classifications of chemotherapeutic agents can be further divided by type of activity or compound.

Typical examples of DNA-interactive agents include the alkylating agents, e.g. Cisplatin, Cyclophosphamide, Altretamine; the DNA strand-breakage agents, such as Bleomycin; the intercalating topoisomerase II inhibitors, e.g., Dactinomycin and Doxorubicin); the nonintercalating topoisomerase II inhibitors such as, Etoposide and Teniposde; and the DNA minor groove binder Plcamydin, wherein

typical alkylating agents include:

| | |
|---|---|
| Nitrogen mustards, such as | Aziridine, such as: |
| carboquone-CAS 24279-91-2 | thiotepa-CAS 52-24-4 |
| chlorambucil-CAS 305-03-3 | Methanesulphonate esters, such as: |
| cyclophosphamide-CAS 6055-19-2 | busulphan-CAS 55-98-1; |
| (Monohydrate) | improsulfan-CAS 1342598-4 |
| ifosfamide-CAS 3778-73-2 | treosulfan-CAS 299-75-2 |
| melphalan-CAS 148-82-3 | Nitroso ureas, such as: |
| multialchilpeptide-CAS 9076-25-9 | carmustine-CAS 154-93-8 |
| mustine-CAS 51-75-2 | chlorozotozin-CAS 54749-90-5 |
| trofosfamide-CAS 22089-22-1 | estramustine-CAS 2998-57-4 |
| uramustine-CAS 66-75-1 | fotemustine-CAS 92118-27-9 |
| Platinum complexes, such as: | lomustine-CAS 13010-47-4 |
| carboplatin-CAS 41575-94-2 | nimustine-CAS 42471-28-3 |
| cisplatin-CAS 15663-27-1 | pipobroman-CAS 54-91-1 |
| enloplatin-CAS 111523-41-2 | prednimustine-CAS 29069-24-7 |
| lobaplatin-CAS 135558-11-1 | ranimusteine-CAS 58994-96-0 |
| miboplatin-CAS 103775-75-3 | semusteine-CAS 13909-09-6 |
| nedaplatin-CAS 95734-82-0 | streptozocin-CAS 18883-66-4 |
| sebriplatin-CAS 110172-45-7 | Bioreductive alkylator, such as: |
| JM-216 | altretamine-CAS 645-05-6 |
| (bisacetoamminedichloro(cyclohexyl- | dacarbazine-CAS 4342-03-4 |
| amine)platium IV) | mitomycin-CAS 50-07-7 |
| Epoxid formation agents, such as: | procarbazine-CAS 671-16-9 |
| mitobronitol-CAS 488-41-5 | porfiromycin-CAS 801-52-5 |
| mitolactol-CAS 10318-26-0 | temozolomide-CAS 85622-93-1; |

and wherein typical examples on DNA strand breaking agents include:

| | |
|---|---|
| bleomycin-CAS 11056-06-7 | tirapazamine-CAS 27314-97-2 |
| peplomycin-CAS 68247-85-8; | |

and wherein typical examples on DNA topoisomerase II inhibitors include:

| | |
|---|---|
| aclarubicin-CAS 57576-44-0 | sobuzoxane-CAS 98631-95-9 |
| crisnatol-CAS 96389-68-3 | topotecan-CAS 123948-87-8 |
| Irinotecan-CAS 97682-44-5; | |

and wherein typical examples on Intercalators include:

| | |
|---|---|
| amsacrine-CAS 51264-14-3, | menogaril-CAS 71628-96-1 |
| dactinomycin-CAS 50-76-0 | mitozantrone-CAS 70476-82-3 |
| daunorubicin-CAS 20830-81-3 | pirarubicin-CAS 72496-41-4 |
| doxorubicin-CAS 23214-92-8 | piroxantrone-CAS 91441-23-5 |
| epirubicin-CAS 56420-45-2 | teloxantrone-CAS 91441-48-4 |
| idarubicin-CAS 58957-92-9 | zorubicin-CAS 54083-22-6; |

and wherein typical examples on nonintercalators include:

| | |
|---|---|
| etoposide-CAS 33419-42-0 | teniposide-CAS 29767-20-2; |

and wherein typical example on DNA minor groove binder includes:
plicamycin-CAS 18378-89-7.
Typical examples on antimetabolites include:

| | |
|---|---|
| Folate antagonists, such as: | Pyrimidine antagonists, such as: |
| edatrexate-CAS 80576-83-6 | azacitidine-CAS 320-67-2 |
| methotrexate-CAS 59-05-2 | azathioprine-CAS 446-86-6 |
| piritrexim-CAS 72732-56-0 | capecitabine-CAS 154361-50-9 |
| trimetrexate-CAS 52128-35-5 | carmofur-CAS 61422-45-5 |
| Floxuridine purine antagonists, e.g.: | cladribine-CAS 4291-63-8 |
| mercaptopurine-CAS 6112-76-1 | flurouracil-CAS 51-21-8 |
| pentostatin-CAS 53910-25-1 | fluorodeoxyuridine-CAS 50-91-9 |
| thioguanine-CAS 154-42-7 | Tegafur-CAS 17902-23-7 |
| Ribonucleotide reductase inhibitors, | sugar modified analogs, such as: |
| such as: | cytarabine-CAS 147-94-4 |
| hydroxyurea-CAS 127-07-1. | decitabine-CAS 2353-33-5 |
| | enocitabine-CAS 55726-47-1 |
| | fludarabine-CAS 21679-14-1 |
| | gemcitabine-CAS 95058-81-4 |

Typical examples on Tubulin Interactive agents include:

| | |
|---|---|
| cyclophosphamide-CAS 6055-19-2 | vinblastine-CAS 865-21-4 |
| decetaxel-CAS 148408-66-6 | vincristine-CAS 57-22-7 |
| lonidamine-CAS 50264-96-2 | vindesine-CAS 53643-48-4 |
| paclitaxel-CAS 33069-62-4 | vinorelbine-CAS 71486-22-1 |
| razoxane CAS 21416-87-5 | |

Typical examples on hormonal agents include:

| | |
|---|---|
| Estrogens, such as: | Progestins, such as: |
| conjugated estrogens-USP | hydroxyprogesterone-CAS 68-96-2 |
| ethinyl estradiol-CAS 57-63-6 | medroxyprogesterone-CAS |
| diethylstilbesterol CAS 56-53-1 | 520-85-4 |
| Chlortrianisen, Idenestrol | megestrol-CAS 3562-63-8 |
| Androgens, such as: | Adrenal corticosteroids such as |
| fluoxymesterone-CAS 76-43-7 | dexamethasone-CAS 50-02-2 |
| methyltestosterone-CAS 58-18-4 | methylprednisolone-CAS 83-43-2 |
| testolactone-CAS 968-93-4 | prednisolone-CAS 50-24-8 |
| testosterone-CAS 58-22-0 | prednisone-CAS 53-03-2 |
| testosterone propionate-CAS 57-85-2 | |
| Antiestrogenic agents, such as: | Luteinizing hormone releasing |
| anastrozole-CAS 120511-73-1 | hormone agents or gonadotropin- |
| droloxifene-CAS 82413-20-5 | releasing hormone antagonists such |
| exemestane-CAS 107868-30-4 | as: |
| fadrozole-CAS 102676-96-0 | goserelin-CAS 65807-02-5 |
| formestane-CAS 566-48-3 | leuprolide-CAS 53714-56-0. |
| letrozole-CAS 112809-51-5 | Antiandrogen agents, such as: |
| rogletimide-CAS 12840-95-7 | bicalutamide-CAS 90357-06-5 |
| tamoxifen-CAS 10540-29-1 | flutamide-CAS 13311-84-7 |
| toremifene-CAS 89778-26-7 | nilutamide-CAS 63612-50-0 |
| vorozole-CAS 129731-10-8 | |
| Antiadrenal agents, such as: | |
| aminoglutethimide-CAS 125-84-8 | |
| mitotane-CAS 53-19-0 | |

The amount and identity of a an anticancer agent that is used with the chemical complexes and compositions according to the invention will vary according to cellular response, patient response and physiology, type and severity of side effects, the disease being treated, the preferred dosing regimen, patient prognosis or other such factors.

It should further be understood that said combination of cysteine derivatives and cimetidine or a derivative thereof, optionally one or more therapeutically active agents may be administered by means of oral, peroral, topical, transdermal, or parenteral administration, or combinations thereof. However, preferable manners of administration are oral and/or topical administration.

### EXAMPLES

The following examples describe the preparation of chemical complexes of the present invention as well as their therapeutic application.

### General Method Example 1

The cimetidine or a derivative thereof as defined herein and the cysteine derivative are dissolved in as little solvent as possible and the solvent is removed by spray drying or freeze-drying. After the solvent is removed the product is a white to yellowish powder. The solvent is water or water with diluted HCl, pH may be in the range from pH 7 to pH 2.

The powder is suitable for being formulated into a pharmaceutical products of any kind. Non-limiting examples of such products are tablets, capsules, ointments and lotions as described above.

Molar ratio of cimetidine or a derivative thereof and a cysteine derivative 1:10000 (mol/mol).

Molar ratio of cimetidine or a derivative thereof and a cysteine
derivative 1:10000 (mol/mol).

| | Cimetidine or a derivative | Cysteine derivative |
|---|---|---|
| No | thereof (1 mol) | (10000 mol) |
| 1. | Cimetidine | N-acetyl-cysteine |
| 2. | Cimetidine HCl | Cystine |
| 3. | Cimetidine | S-ethyl-homocysteine |
| 4. | Cimetidine HCl | N,S-diacetyl-cysteine methylester |

Molar ratio of cimetidine or a derivative thereof and a cysteine
derivative 1:1000 (mol/mol).

| | Cimetidine or a derivative | cysteine derivative |
|---|---|---|
| No | thereof (1 mol) | (1000 mol) |
| 5. | Cimetidine HCl | Cysteine |
| 6. | Cimetidine | Homocysteine |
| 7. | Cimetidine | S-ethyl-cysteine |

Molar ratio of cimetidine or a derivative thereof and a cysteine
derivative 1:100 (mol/mol).

| | Cimetidine or a derivative | cysteine derivative |
|---|---|---|
| | thereof (1 mol) | (100 mol) |
| 8. | Cimetidine | S-ethyl-homocysteine |
| 9. | Cimetidine HCl | N,S-diacetyl-cysteine methylester |
| 10. | Cimetidine | N-acetyl-S-methylcysteine |
| 11. | Cimetidine HCl | S-methylcarboxymethyl-cysteine |

Molar ratio of cimetidine or a derivative thereof and a cysteine
derivative 1:50 (mol/mol).

| | Cimetidine or a derivative | cysteine derivative |
|---|---|---|
| | thereof (1 mol) | (50 mol) |
| 12. | Cimetidine HCl | Cystine |
| 13. | Cimetidine HCl | N,S-diacetyl-cysteine |
| | | methylmethylester |
| 14. | Cimetidine | N-acetyl-S-methylcysteine |
| 15. | Cimetidine HCl | S-carboxymethyl-cysteine |

Molar ratio of cimetidine or a derivative thereof and a cysteine
derivative 6:5 (mol/mol).

| | Cimetidine or a derivative | cysteine derivative |
|---|---|---|
| | thereof (6 mol) | (5 mol) |
| 16. | Cimetidine | N-acetyl-cysteine |
| 17. | Cimetidine HCl | Cystine |
| 18. | Cimetidine | S-ethyl-homocysteine |
| 19. | Cimetidine HCl | N,S-diacetyl-cysteine methylester |

Molar ratio of cimetidine or a derivative thereof and a cysteine
derivative 2:3 (mol/mol).

| | Cimetidine or a derivative | cysteine derivative |
|---|---|---|
| | thereof (2 mol) | (3 mol) |
| 20. | Cimetidine | N-acetyl-cysteine |
| 21. | Cimetidine HCl | Cystine |
| 22. | Cimetidine | S-ethyl-homocysteine |
| 23. | Cimetidine HCl | N,S-diacetyl-cysteine methylester |
| 24. | Cimetidine | N-acetyl-methylcysteine |

Molar ratio of cimetidine or a derivative thereof and a cysteine
derivative 5:1 (mol/mol).

| | Cimetidine or a derivative | cysteine derivative |
|---|---|---|
| | thereof (5 mol) | (1 mol) |
| 25. | Cimetidine | N-acetyl-cysteine |
| 26. | Cimetidine HCl | Cystine |
| 27. | Cimetidine | S-ethyl-homocysteine |
| 28. | Cimetidine HCl | N,S-diacetyl-cysteine methylester |
| 29. | Cimetidine | N-acetyl-S-methylcysteine |
| 30. | Cimetidine HCl | S-carboxymethyl-cysteine |

Molar ratio of cimetidine or a derivative thereof and a cysteine
derivative 50:1 (mol/mol).

| | Cimetidine or a derivative | cysteine derivative |
|---|---|---|
| | thereof (50 mol) | (1 mol) |
| 31. | Cimetidine | N-acetyl-cysteine |
| 32. | Cimetidine HCl | Cystine |
| 33. | Cimetidine | S-ethyl-homocysteine |
| 34. | Cimetidine HCl | N,S-diacetyl-cysteine methylester |

Molar ratio of cimetidine or a derivative thereof and a cysteine
derivative 500:1 (mol/mol).

| | Cimetidine or a derivative | cysteine derivative |
|---|---|---|
| | thereof (500 mol) | (1 mol) |
| 35. | Cimetidine | Cystine |
| 36. | Cimetidine HCl | Homocysteine |
| 37. | Cimetidine | S-ethyl-homocysteine |
| 38. | Cimetidine HCl | N,S-diacetyl-cysteine methylester |
| 39. | Cimetidine | N-acetyl-S-methylcysteine |
| 40. | Cimetidine HCl | S-carboxymethyl-cysteine |

Molar ratio of cimetidine or a derivative thereof and a cysteine
derivative 1000:1 (mol/mol).

| | Cimetidine or a derivative | cysteine derivative |
|---|---|---|
| | thereof 1000 mol) | (1 mol) |
| 41. | Cimetidine | Cysteine |
| 42. | Cimetidine HCl | Cysteine HCl |
| 43. | Cimetidine HCl | Glutathione |
| 44. | Cimetidine | S-ethyl-homocysteine |
| 45. | Cimetidine HCl | N,S-diacetyl-cysteine methylester |
| 46. | Cimetidine HCl | S-carboxymethyl-cysteine |

Molar ratio of cimetidine or a derivative thereof and a cysteine
derivative 10000:1 (mol/mol).

| | Cimetidine or a derivative | cysteine derivative |
|---|---|---|
| | thereof (10000 mol) | (1 mol) |
| 47. | Cimetidine | N-acetyl-cysteine |
| 48. | Cimetidine HCl | Cystine |
| 49. | Cimetidine | S-ethyl-homocysteine |
| 50. | Cimetidine HCl | N,S-diacetyl-cysteine methylester |
| 51. | Cimetidine | N-acetyl-S-methylcysteine |
| 52. | Cimetidine HCl | S-carboxymethyl-cysteine |

Weight ratio Cimetidine or derivative thereof/cysteine
derivative 1:1 (g/g).

| | Cimetidine or a derivative | cysteine derivative |
|---|---|---|
| | thereof (250 mg) | (250 mg) |
| 53. | Cimetidine HCl | Cysteine |
| 54. | Cimetidine | Homocysteine |
| 55. | Cimetidine HCl | Glutathione |
| 56. | Cimetidine | N,S-isobuturyl-cysteine |
| 57. | Cimetidine HCl | S-methyl-cysteine |
| 58. | Cimetidine | S-ethyl-cysteine |

Weight ratio Cimetidine or derivative thereof/cysteine
derivative 2:1 (g/g).

| | Cimetidine or derivative | cysteine derivative |
|---|---|---|
| | thereof (500 mg) | (250 mg) |
| 59. | Cimetidine | Cysteine |
| 60. | Cimetidine | Cystine |
| 61. | Cimetidine HCl | Homocysteine |
| 62. | Cimetidine | N,S-diacetyl-cysteine methylester |
| 63. | Cimetidine HCl | N,S-isobuturyl-cysteine |
| 64. | Cimetidine | S-carboxymethyl-cysteine |
| 65. | Cimetidine HCl | S-ethyl-cysteine |

### General Method Example 2

A quantity of cimetidine or a derivative thereof and the cysteine derivative are transferred to a hard gelatine capsule.

| | |
|---|---|
| Cimetidine or derivative | |
| thereof | cysteine derivative |

Capsule 500 mg, Molar ratio of cimetidine or a derivative thereof
and a cysteine derivative 1:2 (mol/mol).

| | | |
|---|---|---|
| 1. | Cimetidine | N-acetylcysteine |
| | 218 mg | 282 mg |
| 2. | Cimetidine | S-methyl-cysteine |
| | 218 mg | 224.5 mg |
| 3. | Cimetidine | Cysteine S-sulfate |
| | 218 mg | 280.5 mg |
| 4. | Cimetidine HCl | N-acetyl-S-methylcysteine |
| | 246 mg | 252 mg |
| 5. | Cimetidine HCl | S-ethyl-cysteine |
| | 246 mg | 254 mg |
| 6. | Cimetidine HCl | N-acetylcysteine |
| | 246 mg | 246 mg |

Capsule 250 mg, Molar ratio of cimetidine or a derivative thereof
and a cysteine derivative 6:5 (mol/mol).

| | | |
|---|---|---|
| 7. | Cimetidine | Cysteine |
| | 178.5 mg | 71.5 mg |
| 8. | Cimetidine | Cysteine HCl |
| | 178.5 mg | 73.8 mg |
| 9. | Cimetidine | N-acetyl-cysteine |
| | 178.5 mg | 69.7 mg |
| 10. | Cimetidine | Glutathione |
| | 124 mg | 126 mg |
| 11. | Cimetidine HCl | Homocysteine |
| | 179.7 mg | 70.3 mg |
| 12. | Cimetidine HCl | Glutathione |
| | 179.7 mg | 109 mg |
| 13. | Cimetidine HCl | Cysteine methylester HCl |
| | 179.7 mg | 74.5 mg |
| 14. | Cimetidine HCl | S-ethyl-cysteine |
| | 174.7 mg | 75.3 mg |

### Example 3

Study Object

The effect of a complex of the invention is tested on tumor progression in SCID mice xenografted with SW620 colorectal cancer cells. The aim of the study is to produce growth curves of the grafted tumor and to monitor the effect of the complex of the invention on the growth curves.

Test Compounds

The complex according to compound 20 of example 1 is tested.

Dosing Pattern

Compound 20 is dissolved in drinking water. The test compound is administered at a concentration of 1.5 mg/ml and the solution is available ad libitum in the entire study period. Assuming a daily water intake of 2 ml and an average body weight of 20 g per mouse the administered amount of test compound corresponds to a daily dose of 150 mg/kg.

Animals

In this study, female SCID mice with an age of 6 weeks are used. Ten mice are included per group. The mice are caged in standard cages-at a temperature of 21° C.-23° C. controlled via the ambient ventilation system in the laboratory. Light cycle is 12-hour dark and 12-hour light (lights on 06.00). Diet is Altromin 1314 special formulation, Produced by Altromin Denmark, Chr. Pedersen A/S, 4100 Ringsted, Denmark. Water is acidified with citric acid. Diet and water is administered ad libitum.

Method

The mice are randomised to test groups of ten mice. After one week of acclimatisation each mouse is injected subcutaneously with 2.0×10⁵ cells contained in 0.1 ml of phosphate buffered saline (PBS). The cell line used is SW620, which is a standard human colorectal cancer cell. Tumors are then allowed to grow for 40 days. Tumor diameters are measured in two dimensions using a digital slide gauge. Tumor diameters are measured at days 9, 14, 19, 22, 26, 30, 32, 36 and 40.

Findings and Interpretation

Mean tumor sizes in the vehicle treated group was 485 mm³ at day 40. Tumor growth in the group treated with the complex according to the invention was inhibited over the entire study period. Maximal inhibition was observed in the exponential growth phase. An inhibition of tumor volume of 89% (p<0.05, Mann-Whitney), 73% (p<0.05, Mann-Whitney) and 47% (p<0.05, Mann-Whitney) was observed at days 22, 30 and 32 respectively.

The study demonstrated a surprising and highly significant tumor inhibiting effect of the complex of the invention.

### Example 4

Study Object

The effect of a complex of the invention and the effect of the components of the complex is tested on tumor growth in BALB/c mice grafted with syngenic CT26 colorectal cancer cells. The aim of the study is to compare the effect on tumor growth of the complex of the invention versus the effect of the two components of the complex individually. The results are obtained from two individual studies under identical conditions as described below.

Test Compounds

The complex according to compound 20 of example 1 is tested.

Dosing Pattern

Compound 20, cimetidine and N-acetyicysteine is dissolved in drinking water. Compound 20 is administered at a concentration of 1.5 mg/ml. Cimetidine and N-acetyl cysteine are administered at concentrations of 0.75 mg/ml respectively. Thus, Cimetidine and N-acetyl cysteine are administered individually at a concentration corresponding to the amount of each substance in the 1,5 mg/ml concentration of Compound 20. The solutions are available ad libitum in the entire study period. Assuming a daily water intake of 2 ml and an average body weight of 20 g per mouse the administered amount of compound 20 corresponds to a daily dose of 150 mg/kg. Based on the same assumption the administered amounts of cimetidine and N-acetyl cysteine correspond to a daily dose of 75 mg/kg respectively. The doses of cimetidine and N-acetylcysteine are selected to correspond to the doses of each individual compound obtained by administration of 150 mg/mg of compound 20.

Animals

In these studies, female BALB/c mice with an age of 6 weeks are used. Ten mice are included per group. The mice are caged in standard cages at a temperature of 21° C.-23° C. controlled via the ambient ventilation system in the laboratory. Light cycle is 12-hour dark and 12-hour light (lights on 06.00). Diet is Altromin 1314 special formulation, Produced by Altromin Denmark, Chr. Pedersen A/S, 4100 Ringsted, Denmark. Water is acidified with citric acid. Diet and water is administered ad libitum.

Method

The mice are randomised to test groups of ten mice. After one week of acclimatisation each mouse is injected subcutaneously with appr. 1.0×10⁵ cells contained in 0.1 ml of phosphate buffered saline (PBS). The cell line used is CT26, which is a standard syngenic colorectal cancer cell. Tumors are then allowed to grow for 33 days. Tumor diameters are measured in two dimensions using a digital slide gauge.

Findings and Interpretation

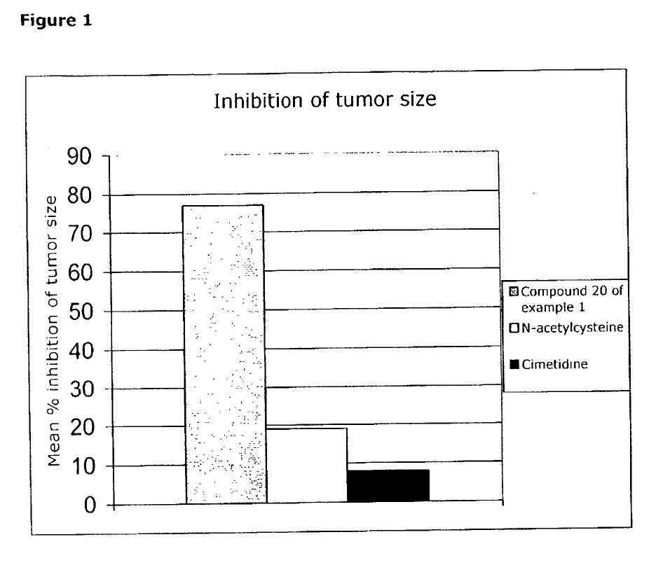

The results are shown in FIG. 1.

Mean tumor sizes in the vehicle treated groups were 398 and 450 mm³ at day 33. Tumor growth in the group treated with the complex according to the invention was inhibited by 77% (p<0.05, Mann-Whitney) at day 33. In the group treated with cimetidine tumor growth was inhibited by 8% (not significant) and in the N-acetyl cysteine treated group inhibition was 19% (not significant).

Overall Thiomestat yielded a 185% higher inhibition of tumor size than the sum of the inhibition of the components administered individually. This finding indicates a synergistic effect and that Thiomestat is pharmacodynamically superior to cimetidine and N-acetyicysteine in this test model system.

1. A substance consisting of a chemical complex comprising:
i) a cysteine derivative of Formula I, stereoisomers thereof and/or salts thereof, 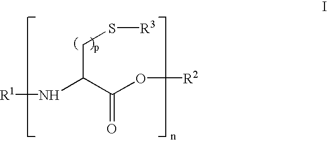
wherein n is an integer from 1 to 6;
p is a whole number selected from the group consisting of 0, 1 and 2;
R¹ is a monoradical selected from the group consisting of hydrogen, halogen, sulphate, optionally substituted C₁-C₈-acylene, optionally substituted C₁-C₈-alkylene, optionally substituted C₃-C₇-cycloalkylene, optionally substituted C₂-C₈-alkenylene and optionally substituted C₂-C₈-alkynylene;
R² is a monoradical selected from the group consisting of hydrogen, halogen, sulphate, optionally substituted C₁-C₈-alkylene, optionally substituted C₃-C₇-cycloalkylene, optionally substituted C₂-C₈-alkenylene and optionally substituted C₂-C₈-alkynylene; and
R³ is a monoradical selected from the group consisting of hydrogen, sulphate, optionally substituted C₁-C₈-alkylene, optionally substituted C₂-C₈-alkenylene, optionally substituted C₂-C₈-alkynylene, optionally substituted arylene, optionally substituted heteroarylene and 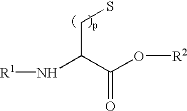
wherein p, R¹ and R² are independently selected from their groups as defined above; and
ii) cimetidine or a derivative thereof according to Formula II, and/or salts thereof, 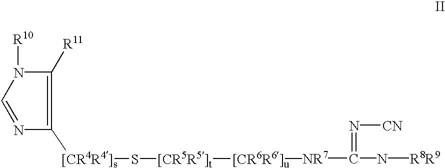
wherein s is a whole number from 1-3, t is a whole number from 0-2 and u is a whole number from 1-2;
R⁴, R^{4′}, R⁵, R^{5′}, R⁶, R^{6′} are each a monoradical independently selected from the group consisting of hydrogen, halogen, hydroxyl, C₁-C₆-alkylene, C₁-C₈-acylene and arylene;
R⁷, R⁸, R⁹, R¹⁰ are each a monoradical independently selected from the group consisting of hydrogen, nitro, C₁₋₆-alkylene, C₂-C₆-alkenyl, C₁₋₈-acylene and arylene; and
R¹¹ is a monoradical independently selected from the group consisting of hydrogen, halogen, hydroxy, C₁-C₆-alkylene, C₁-C₈-acylene and arylene.

2. The substance according to claim 1, wherein n is selected from the group consisting of 2 and 3; and R³ is a monoradical selected from the group consisting of hydrogen, sulphate, optionally substituted C₁-C₈-alkylene, optionally substituted C₂-C₈-alkenylene, optionally substituted C₂-C₈-alkynylene, optionally substituted arylene and optionally substituted heteroarylene.

3. The substance according to claim 1, wherein n is 1 and R³ is 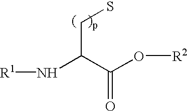
and wherein R¹ and R² are independently selected from their groups as defined above.

4. The substance according to claim 1, wherein the cysteine derivative of Formula I is selected from the group consisting of cysteine, N-acetyl-cysteine, cystine, homocysteine, cysteine methylester, S-ethyl-cysteine, N,S-isobuturyl-cysteine, S-carboxymethyl-cysteine, S-ethyl-homocysteine, S-methyl-cysteine, cysteine S-sulfate, N,S-diacetyl-cysteine methylester, N-acetyl-S-methylcysteine, glutathione, stereoisomers thereof, salts thereof and mixtures thereof.

5. The substance according to any one of claims 1 to 4, wherein the cimetidine or a derivative thereof, or a salt thereof is selected from the group consisting of cimetidine and salts selected from the group consisting of cimetidine hydrochloride, cimetidine hydrobromide, cimetidine acetate, cimetidine ascorbate and cimetidine benzoate.

6. The substance according to any one of claims 1 to 5, wherein the cimetidine or a derivative thereof and the cysteine derivative are present in a molar ratio of between about 1:10000 to 10000:1, such as about 1:1000 to 1000:1, preferably about 1:100 to 100:1, such as about 1:10 to 10:1, more preferably from about 1:5 to 5:1, such as about 1:2 to 2:1.

7. The substance according to any one of claims 1 to 6, wherein the cimetidine or a derivative thereof and the cysteine derivative are present in a mass ratio of between about 1:10000 to 10000:1 such as, about 1:1000 to 1000:1, preferably about 1:100 to 100:1, such as about 1:10 to 10:1, more preferably from about 1:5 to 5:1, such as about 1:2 to 2:1.

8. The substance according to any one of claims 1 to 7, wherein the complex further comprises one or more therapeutically active agents.

9. The substance according to claim 8, wherein the one or more therapeutically active agent is an anticancer agent.

10. The substance according to claim 9, wherein the anticancer agent is selected from the group consisting of DNA-interactive agents, antimetabolites, tubulin-interactive agents hormonal agents, protease inhibitors, cyclooxygenase inhibitors, nuclear factor kappa B inhibitors and 3-hydroxy-3-methylglutaryl-coenzyme A (HMG-CoA) inhibitors and vitamin D derivatives and vitamin D analogs.

11. A composition comprising
i) a complex comprising a cysteine derivative of Formula I, stereoisomers thereof and/or salts thereof, said Formula I as defined in claim 1, and cimetidine or a derivative thereof of Formula II, and/or salts thereof, said Formula II as defined in claim 1; and
ii) one or more acceptable excipient(s) or carrier(s).

12. A composition comprising
i) a cysteine derivative of Formula I, stereoisomers thereof and/or salts thereof, wherein said Formula I as defined in claim 1;
ii) cimetidine or a derivative thereof according to Formula II, and/or salts thereof, said Formula II as defined in claim 1; and
iii) one or more acceptable excipient(s) or carrier(s).

13. The composition according to any one of claims 11 or 12, wherein n is selected from the group consisting of 2 and 3; and R³is a monoradical selected from the group consisting of hydrogen, sulphate, optionally substituted C₁-C₈-alkylene, optionally substituted C₂-C₈-alkenylene, optionally substituted C₂-C₈-alkynylene, optionally substituted arylene and optionally substituted heteroarylene.

14. The composition according to any one of claims 11 or 12, wherein n is 1 and R³ is 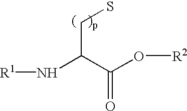
and wherein R¹ and R² are independently selected from their groups as defined above.

15. The composition according to any one of claims 11 or 12, wherein the cysteine derivative of Formula I is selected from the group consisting of cysteine, N-acetyl-cysteine, cystine, homocysteine, cysteine methylester, S-ethyl-cysteine, N,S-isobuturyl-cysteine, S-carboxymethyl-cysteine, S-ethyl-homocysteine, S-methyl-cysteine, cysteine S-sulfate, N,S-diacetyl-cysteine methylester, N-acetyl-S-methylcysteine, glutathione, stereoisomers thereof, salts thereof and mixtures thereof.

16. The composition according to any one of claims 11 to 15, wherein the cimetidine or a derivative thereof, or a salt thereof is selected from the group consisting of cimetidine and salts selected from the group consisting of cimetidine hydrochloride, cimetidine hydrobromide, cimetidine acetate, cimetidine ascorbate and cimetidine benzoate.

17. The composition according to any one of claims 11 to 16, wherein the cimetidine or a derivative thereof and the cysteine derivative are present in a molar ratio of between about 1:10000 to 10000:1, such as about 1:1000 to 1000:1, preferably about 1:100 to 100:1, such as about 1:10 to 10:1 e.g. about 1:5 to 5:1, such as about 1:2 to 2:1.

18. The composition according to any one of claims 11 to 17, wherein the cimetidine or a derivative thereof and the cysteine derivative are present in a mass ratio of between about 1:10000 to 10000:1 such as, about 1:1000 to 1000:1, preferably about 1:100 to 100:1, such as about 1:10 to 10:1 e.g. about 1:5 to 5:1, such as about 1:2 to 2:1.

19. The composition according to any one of claims 11 to 18, further comprising one or more therapeutically active agents.

20. The composition according to claim 19, wherein the one or more therapeutically active agents is an anticancer agent.

21. The composition according to claim 20, wherein the anticancer agent is selected from the group consisting of DNA-interactive agents, antimetabolites, tubulin-interactive agents hormonal agents, protease inhibitors, cyclooxygenase inhibitors, nuclear factor kappa B inhibitors and 3-hydroxy-3-methylglutaryl-coenzyme A (HMG-CoA) inhibitors and vitamin D derivatives and vitamin D analogs.

22. The composition according to any one of claims 11 to 21, formulated for administration selected from the group consisting of peroral, oral, topical, transdermal, and parenteral administration.

23. The composition according to claim 22, formulated for administration selected from the group consisting of peroral and topical administration.

24. The composition according to any one of claims 11 to 23, formulated in a form selected from the group consisting of a solid, a semi-solid, a suspension and an emulsion.

25. A method for treating cancer in a mammal, comprising administration to a mammal of an effective amount of a combination of cimetidine or a derivative thereof of Formula II as defined in claim 1, and/or salts thereof; and a cysteine derivative of formula I as defined in claim 1, stereoisomers thereof and/or salts thereof.

26. The method according to claim 25, wherein the cancer is selected from the group of cancer in the gastrointestinal system, metastatic cancers and invasive cancers.

27. The method according to claim 26, wherein the cancer of the gastrointestinal system is selected from the group of colon cancer, rectal cancer, colorectal cancer, pancreatic cancer, stomach (gastric) cancer, oesophageal cancer, liver cancer or bladder cancer.

28. The method according to claim 26, wherein the metastatic cancers and invasive cancers cancer is selected from the group of breast cancer, cancer of the male and female genital tract, cancer of the thymus, lung, stomach, small intestine, prostate, adrenal gland, pancreas, colon, lymphoid tissue, liver, brain, salivary gland, spleen and skin.

29. A method for immunomodulation in a mammal, comprising administration to said mammal of an effective amount of a combination of cimetidine or a derivative thereof of Formula II as defined in any one of claims 1 to 3 and a cysteine derivative as defined in any one of claims 1 to 3, or a chemical complex comprising said combination or said salts to said mammal.

30. The method according to claim 29, wherein immunomodulating activity relates to the suppression of inflammatory reactions such as treatment of diseases and disorders, or symptoms associated therewith, selected from the group consisting of hypersensitivity skin disease, atopic eczema, contact dermatitis, seborrhoeic eczema, psoriasis, IgE mediated allergic reactions, asthma, allergic rhinitis, anaphylaxis, autoimmune disease, chronic inflammatory disease, Crohn's disease, ulcerative colitis, rheumatoid arthritis, gout, osteoarthritis and pain.

31. The method according to any one of claims 25 or 29, wherein the cysteine derivative is selected from the group consisting of cysteine, N-acetyl-cysteine, cystine, homocysteine, cysteine methylester, S-ethyl-cysteine, N,S-isobuturyl-cysteine, S-carboxymethyl-cysteine, S-ethyl-homocysteine, S-methyl-cysteine, cysteine S-sulfate, N,S-diacetyl-cysteine methylester, N-acetyl-S-methylcysteine, glutathione, stereoisomers thereof, salts thereof and mixtures thereof.

32. The method according to any one of claims 25 or 29, wherein the cimetidine or a derivative thereof, or a salt thereof is selected from the group consisting of cimetidine and cimetidine salts selected from the group consisting of cimetidine hydrochloride, cimetidine. hydrobromide, cimetidine acetate, cimetidine ascorbate and cimetidine benzoate.

33. The method according to any one of claims 25 or 29, wherein the combination of cimetidine or a derivative thereof and the cysteine derivative, is a substance consisting of a chemical complex as defined in any one of claims 1 to 10.

34. The method according to any one of claims 25 or 29, wherein the combination of cimetidine or a derivative thereof and the cysteine derivative is a composition as defined in any one of claims 11 to 24.

35. The method according to any one of claims 25 or 29, further comprising the administration of one or more therapeutically active agents.

36. The method according to claim 35, wherein the one or more therapeutically active agents is an anticancer agent.

37. The method according to claim 36, wherein the anticancer agent is selected from the group consisting of DNA-interactive agents, antimetabolites, tubulin-interactive agents hormonal agents, protease inhibitors, cyclooxygenase inhibitors, nuclear factor kappa B inhibitors and 3-hydroxy-3-methylglutaryl-coenzyme A (HMG-CoA) inhibitors and vitamin D derivatives and vitamin D analogs.

38. The method according to any one of claims 25 or 29, wherein said combination of cimetidine or a derivative thereof and the cysteine derivative is administered by means of peroral, oral, topical, transdermal, or parenteral administration, or combinations thereof.

39. The method according to any one of claims 25 or 29, wherein the combination of cimetidine or a derivative thereof and the cysteine derivative, are together comprised in a single formulation.

40. The method according to any one of claims 25 or 29, wherein the combination of cimetidine or a derivative thereof and the cysteine derivative are each individually comprised in separate formulations.
